(19) United States
(12) Patent Application Publication
(10) Pub. No.: US 2009/0064996 A1
(43) Pub. Date: Mar. 12, 2009

(54) Duo chamber

(76) Inventor: Melvin S. Rosh, New York, NY (US)

Correspondence Address: MELVIN S. ROSH, 150 E. 93 STREET, APT. 8-C, NEW YORK, NY, 10128, US

(21) Appl. No.: 11/900,020

(22) Filed: Sep. 10, 2007

(51) Int. Cl. A61M 11/00

(52) U.S. Cl. 128/200.23

(57) ABSTRACT

The DUOCHAMBER is a “spacer” devise used to efficiently deliver aerosol medication to the lung obstructed patient. Since most of these patients require at least two different inhalant medication, the DUOCHAMBER allows two MDI canisters of medication to be ready to be used at any time, under the direction of the patient's primary care giver.

The DUOCHAMBER is small, efficient and conveniently portable. The parts are easily dismantled for cleaning or replacement of parts.

### BACKGROUND OF THE INVENTION

More than twenty three million Americans suffer with a medical condition known as ASTHMA and millions more suffer with CHRONIC OBSTRUCTIVE LUNG DISEASE, which are characterized by wheezing, shortness of breath and coughing. Numerous medications have been formulated to treat the conditions.

The most popular form of treatment is inhaled medication via a metered dose inhalation (MDI) canister. The medication propels into the throat at high speeds during inspiration. Much of it is lost as it bounces off the posterior pharyngeal wall and out the mouth. To overcome this problem, the medication can be expelled into a chamber, known as a “spacer”, and then inhaled into the lungs.

Many types of inhaled medication are used. Cortisone is an anti-inflammatory agent. Albuterol compounds counteract the bronchospasm in the lungs. Chromolyn inhalation helps prevent chronic asthma. Other inhalation medications are also available. However, less than twenty five percent of asthmatics and chronic obstructive lung disease patients are successfully treated with only one medication. The majority of patients with these conditions relies on at least two inhalation preparations.

Inhalation spacers have been developed which allow the patient to use only one canister of medication at a time. The DUOCHAMBER holds two canisters and each medication can be inserted into the chamber at the same time without needing to remove the first canister used in order to insert the second one.

For the patient on only one medication, both canisters can be of the same medication, so that when one is empty, the patient has a spare ready to be used when needed.

### CURRENT US CLASS

### Field of Search

| | | |
|---|---|---|
| 5,427,089 | Jun. 27, 1995 | Kraemer |
| 6,470,882 | Sep. 29, 1997 | Newhouse |
| 5,792,057 | Aug. 11, 1998 | Rubsamen |
| 6,202,643 | Mar. 20, 2001 | Sladek |
| 6,039,042 | Mar. 21, 2001 | Sladek |
| 6,425,392 | Jul. 30, 2002 | Sosiak |
| 6,550,473 | Apr. 22, 2003 | Sladek |
| 6,584,969 | Jul. 1, 2003 | Farmer |
| 6,679,252 | Jan. 20, 2004 | Sladek |

### BRIEF SUMMARY OF THE INVENTION

Inhalation medications is best administered by means of a spacer devise. The use of a spacer makes delivery of the medication far more efficient. The DUOCHAMBER is a small spacer that is conveniently carried and allows the patient to have two inhalation medications available when needed. The primary care giver instructs the patient under what circumstances and at which times the medication(s) are to be taken. Seventy to seventy five percent of asthmatics and patients with other chronic pulmonary diseases require two different inhalation medications to control their illness.

After the two medication canisters are in their holders, the appropriate canister is pushed and the spacer fills with the medication. As the particles spray into the chamber of the spacer, the latex-free rubber valve closes the opening on the air intake tube, preventing loss of medication, and the latex-free rubber wing projection is pushed upward, indicating that the canister has discharged. If the canister is empty, the wing projection will not move.

The patient is instructed to take three deep breaths to open the airways and then after the last deep expiration, to hold his breath, remove the mouthpiece cap and quickly place his mouth over the mouthpiece. The patient, without removing his lips from the mouthpiece, takes in three deep breaths, while exhaling through his nostrils or through the lateral sides of his mouth around the mouthpiece. As the breath in inhaled, the rubber valve on the window of the air intake tube opens to allow air to rush into the spacer, so that the medication can be inhaled into the lungs. After inhalation, while the patient is exhaling through the nostrils or sides of the mouth, the rubber side flap on the central tube acts like a valve and closes the window of the air intake tube, preventing minimal loss of medication. After three breaths, most of the medication in the chambers will have been delivered into the pulmonary system. The mouthpiece cap is repositioned and the unit is ready for the administration of the next dose.

### BRIEF DESCRIPTIONS OF THE SEVERAL VIEW OF THE DRAWINGS

FIG. 1 DUOCHAMBER

Displays DUOCHAMBER in entirety without the metered dose inhaler (MDI) canisters. The canisters (not shown) fit into the openings of the MDI canister holders (107) in the base (106) of the bottom chamber (101). The opening of the air intake tube (109) can be found between the two MDI canister holders (107) on the base (106).

The DUOCHAMBER consists oftwo parts. (a) A bottom chamber (101) and (b) a top chamber (102). Both chambers fit together and sealed with a latex-free rubber gasket (112). The oval mouth piece (104) is at the top of the top chamber (102). A mouth piece cover (105) seals the mouth piece 104) with the aid of a latex free rubber gasket (112).

FIG. 2 AIR INTAKE TUBE

The air intake tube (108) is attached to the base (106) of the bottom chamber (101), where it is open ended (109). The upper end (110) of the air intake tube (108) is sealed.

Open windows (111) are located on both lateral sides of the air intake tube (108).

FIG. 3 RUBBER SLEEVE WITH WINGS

Over the air intake tube (108) fits a removable latex-free rubber sleeve (118) with movable side flaps (113) which cover the open windows (111) of the air intake tube (108) and act as valves. Two movable latex-free rubber wings (114) project laterally from the upper end of the rubber sleeve (118).

FIG. 4 MDI CANISTER HOLDER

A MDI Canister (115) is shown in the canister holder (107). When the canister is pushed into the canister holder (107) the nozzle (116) of the canister is depressed and a dose of medication is pushed out of the spray opening (117) into the chamber of the DUOCHAMBER.

### DETAILED DESCRIPTION OF THE INVENTION

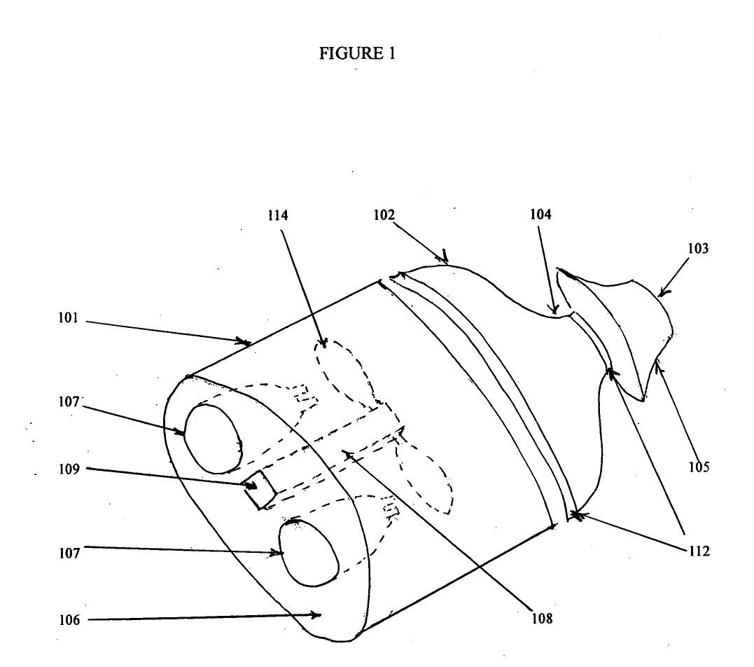

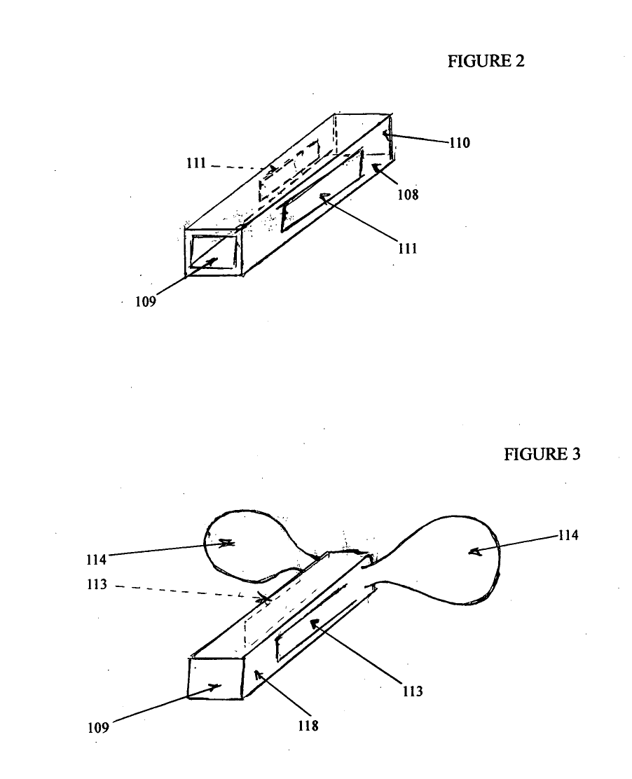

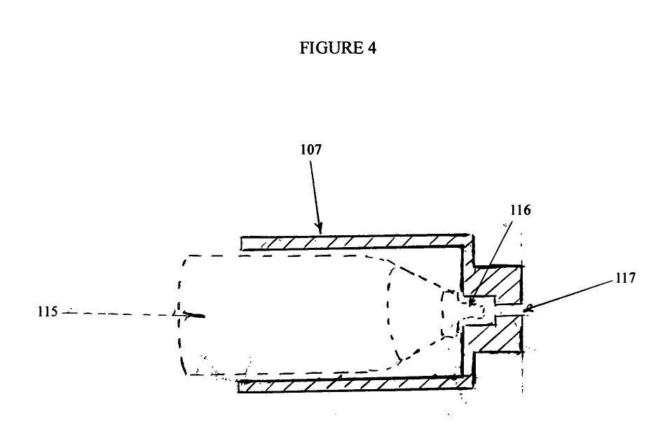

Referring to FIG. 1, the DUOCHAMBER is composed of three parts. (1) a bottom chamber (101) (2) a top chamber (102) and (3) a mouthpiece cover (103). The base (106) of the bottom chamber (101) has three openings. The center opening (109) leads into the air intake tube (108). The two larger openings on either side of the air intake tube (108) lead into the holders (107) for the MDI canisters of inhalation medication. When the canister is pushed down into the holder (107), medication is released and then the canister is readied for the next dose of medication. The DUOCHAMBER can be easily taken apart for cleaning.

FIG. 2 shows the air intake tube (108) which has two open windows (111) on each lateral side and is sealed at the upper end (110). The open lower end (109) of the air intake tube (108) is attached to the base (106) of the bottom chamber (101)

FIG. 3 shows the latex-free rubber sleeve (118) which slides over the air intake tube (108). The sleeve has two side flaps (113) which fit over the open windows (111) of the air intake tube (108). When the MDI canister injects medication into the spacer, the side flaps (113) close the windows (111) of the air intake tube (108). Upon inspiration by the patient, the slide flaps (113) open the windows (111) allowing air to come into the spacer. At the upper end of the rubber sleeve (118) are two lateral rubber “wing” projections (114), which move when medication is released from the MDI canisters or when the patient takes an inspiration. The rubber sleeve (118) is removable for cleaning or replacing.

FIG. 4 depicts a MDI canister (115) in the canister holder (107). An enlargement of the nozzle (116) area is displayed. When the MDI canister is pushed into the holder, the nozzle (116) is depressed, releasing the aerosol medication through the spray opening (117) into the spacer. The two holders (107) are made to fit a standard MDI canister (115).

The assembled DUOCHAMBER is approximately five inches in length and can be easily carried in a pocket book or in a belt pouch. The bottom chamber measures three inches; the top chamber, two inches and the mouth piece cover, three quarters of an inch. Latex-free rubber gaskets seal the top and bottom chambers as well as the top chamber and the mouth piece cover in order to prevent loss of medication.

I claim:

1. A DUOCHAMBER is a medication inhalation apparatus (spacer) which holds two MDI canisters of inhalation medication.

2. The propelled particles from the MDI canister is dispersed throughout the chambers of the spacer as they contact the latex-free rubber wing-like projections attached to the latex-free rubber sleeve on the air intake tube; movement of the rubber wing-like projections indicate release of medication from the MDI canister and proper inhalation by the user.

3. Latex-free rubber valves on the air intake tube control the direction of air flow into the chamber.
